(19) United States
(12) Patent Application Publication
(10) Pub. No.: US 2007/0048367 A1
(43) Pub. Date: Mar. 1, 2007

(54) Herbal composition for treating morning sickness

(75) Inventor: Roshan Kaderali, San Rafael, CA (US)

Correspondence Address: JULIA CHURCH DIERKER;DIERKER & ASSOCIATES, P.C., 3331 W. BIG BEAVER RD. SUITE 109, TROY, MI, 48084-2813, US

(73) Assignee: MOM Enterprises, Inc.

(21) Appl. No.: 11/510,343

(22) Filed: Aug. 25, 2006

Related U.S. Application Data
(60) Provisional application No. 60/713,035, filed on Aug. 31, 2005.

(51) Int. Cl. A61K 36/906; A61K 9/64; A61K 36/73; A61K 31/525; A61K 31/4415

(52) U.S. Cl. 424/456; 424/756; 424/765; 514/251; 514/350

(57) ABSTRACT

An herbal composition for treating morning sickness includes a mixture of vitamin B6, folic acid, ginger root extract, and red raspberry leaf.

### CROSS-REFERENCE TO RELATED APPLICATIONS

This application claims the benefit of U.S. Provisional Patent application Ser. No. 60/713,035, filed Aug. 31, 2005

### BACKGROUND

The present disclosure relates generally to herbal compositions, and more particularly to an herbal composition for treating morning sickness.

Over half of the women who become pregnant experience morning sickness. Some of the symptoms of morning sickness include vomiting and/or nausea. Morning sickness may occur at any time throughout the day. Some women experience morning sickness for a short period of time, while others experience morning sickness throughout their entire pregnancy. Generally, the degree of severity of the morning sickness varies from woman to woman. As the risks of taking medications (including over-the-counter medications) while pregnant may be unknown, women may be advised against taking such medications for treating morning sickness. As such, the remedies available for treating morning sickness may be limited.

As such, it would be desirable to provide an herbal composition for treating the symptoms of morning sickness.

### SUMMARY

An herbal composition for treating morning sickness includes a mixture of vitamin B6, folic acid, ginger root extract, and red raspberry leaf.

### DETAILED DESCRIPTION

Embodiments of the herbal composition disclosed herein are capable of substantially reducing or eliminating nausea and/or vomiting often associated with morning sickness. The herbal composition ingredients work together to substantially effectively treat the symptoms of morning sickness. Without being bound to any theory, it is believed that the combination of active ingredients described herein has a synergistic effect in embodiment(s) of the present composition. Generally, an embodiment of the herbal composition includes vitamin B6, folic acid, ginger root extract, and red raspberry leaf. It is to be understood that each of the ingredients contributes to reducing the symptoms of morning sickness and/or to the welfare of the mother and/or to the welfare of the unborn baby.

In some embodiments, the herbal composition includes 100% of naturally occurring ingredients. In other embodiments, the herbal composition includes some naturally occurring ingredients and some synthetic ingredients.

Vitamin B6 (pyridoxine hydrochloride) is effective in reducing nausea, vomiting, and motion sickness. Vitamin B6 has also been found to strengthen a user's immune system and stabilize a user's mood. It is to be understood that the vitamin B6 in embodiments of the composition may be substituted with a suitable pharmaceutical, herbal, and/or vitamin alternative(s). Some non-limitative examples of such alternatives to vitamin B6 include doxylamine succinate, dicyclomine, ondansetron, prochloroperazine, ginger root extract, peppermint, a variety of Chinese herbs, or the like, or combinations thereof. In an embodiment, about 1 tbsp (about 15 ml) of the composition includes from about 10 mg to about 35 mg of vitamin B6. In an alternate embodiment, about 1 tbsp (about 15 ml) of the composition includes from about 15 mg to about 30 mg of vitamin B6. In a non-limitative example, the composition has about 25 mg of vitamin B6 per 1 tbsp (15 ml).

Folic acid is a nutrient that is substantially essential for normal fetal development, and is believed to be effective in the prevention of birth defects. In an embodiment, about 1 tbsp (about 15 ml) of the composition includes from about 100 mcg to about 800 mcg of folic acid. In an alternate embodiment, about 1 tbsp (about 15 ml) of the composition includes from about 200 mcg to about 500 mcg of folic acid. In a non-limitative example, the composition has about 200 mcg of folic acid per 1 tbsp (15 ml).

Ginger root extract (zingiber officinale) is generally considered to be an expectorant, antiemetic, analgesic, antispasmodic, carminative, diaphoretic, and/or rubefacient. Ginger root extract is effective in reducing, for example, nausea, vomiting, motion sickness, and colic, and may aid in digestion. It is to be understood that the ginger root extract in an embodiment of the composition may be substituted with a suitable pharmaceutical, herbal, and/or vitamin alternative(s). Some non-limitative examples of such alternatives to ginger root extract include doxylamine succinate, dicyclomine, ondansetron, prochloroperazine, vitamin B6, peppermint, catnip, licorice root, cayenne, a variety of Chinese herbs, or the like, or combinations thereof. In an embodiment, about 1 tbsp or about 15 ml of the composition includes from about 10 mg to about 100 mg of ginger root extract. In a non-limitative example, the composition has about 50 mg of ginger root extract per 1 tbsp (15 ml).

Red raspberry leaf (rubus idaeus) is generally an anti-abortifacient, antiseptic, antispasmodic, astringent, hemostatic, hormonal regulator, hypotensive, kidney tonic, nutritive, parturient, prostate tonic, uterine tonic, and yin tonic. Red raspberry leaf may be effective in reducing, for example, nausea and diarrhea, and for promoting, for example, uterine health (e.g. strengthening the pelvic and/or uterine muscles). In an embodiment, about 1 tbsp or about 15 ml of the composition includes from about 5 mg to about 35 mg of red raspberry leaf. In a non-limitative example, the composition has about 20 mg of red raspberry leaf per 1 tbsp (15 ml).

In a non-limitative embodiment, about 1 tbsp (about 15 mL) of the composition is about one serving. However, it is to be understood that a single serving quantity may vary.

It is to be understood that the composition may also include one or more other additional ingredients. Examples of suitable additional ingredients include, but are not limited to water (e.g., purified water), fructose, natural flavors, citric acid, coloring (e.g., beta carotene coloring), potassium sorbate, sodium benzoate, chamomile (i.e., a gastro-intestinal relaxant and anti-spasmodic), peppermint, spearmint, papaya, catnip, squaw vine, rice flour, magnesium stearate, silica dioxide, and/or combinations thereof. Generally, rice flour, magnesium stearate, silica dioxide, and/or the like are included in a capsule form of the composition. In a non-limitative example, one or more of the additional ingredients may act as a carrier or vehicle for the active ingredients of the herbal composition.

The herbal composition may be made in the form of liquids, powders, tablets, teas, capsules, and/or gelatins. In an embodiment of the method, the ingredients are mixed using automated manufacturing equipment. It is to be understood however, that any suitable manufacturing technique may be selected, depending, at least in part, on the desired form of the herbal composition.

It is to be understood that the composition according to embodiment(s) herein may be made to have any flavor (e.g., peach, ginger, lemon, berry, orange, combinations thereof, etc.), as desired.

To further illustrate embodiment(s) of the present disclosure, the following example and experimental data are given. It is to be understood that this example and information are provided for illustrative purposes and are not to be construed as limiting the scope of embodiment(s) of the present disclosure.

### EXAMPLE

The composition is a liquid having a total volume of about 120 ml (+/−2 ml). The composition includes about 25 mg vitamin B6 and about 200 mcg folic acid per 15 ml of the composition. The composition further includes about 50 mg ginger root extract and about 20 mg red raspberry leaf per 15 ml of composition. The specific gravity of the composition ranges from about 1.000 to about 1.200, and the pH ranges from about 3 to about 5. The composition smells like ginger and peach, and has a relatively sweet ginger and peach taste. The color of the composition is an orange/brown color, and the appearance is substantially opaque.

### EXPERIMENTAL DATA

Doctors across the United States were each sent four bottles of the composition described herein, and were invited to allow their pregnant patients to try the composition as a remedy for morning sickness. The same doctors were also sent a survey inquiring about the patients'experience with the composition, the other remedies used, and how the composition disclosed herein compared with the other remedies. Over about a six month time period, 50 doctors returned the surveys, which, in total, included results for 169 patients.

Most of the patients surveyed were between the ages of 18 and 30, and most were between 6 and 10 weeks pregnant. Overall, 12% of the patients (20 patients) were less than 18 years old, 66% of the patients (112 patients) were between the ages 18 and 30, and 22% of the patients (37 patients) were over 30 years old. 63% of the patients (106 patients) were between 6 and 10 weeks pregnant, 24% of the patients (41 patients) were greater than 10 weeks pregnant, 9% of the patients (16 patients) were less than 6 weeks pregnant, and 4% of the patients (6 patients) did not respond to the question regarding pregnancy duration.

The doctors were initially asked to rate the degree of the patient's morning sickness prior to providing her with the composition disclosed herein or any other morning sickness remedy. The question asked the doctors to rate the symptoms using the following 1 to 5 scale: where 1=not a problem at all, and 5=completely debilitating. Table 1 illustrates the results of the doctors' answers.

TABLE 1
Degree of Patients' Symptoms Prior to Morning Sickness Remedy*

| Response | Number of Patients | Percent of Patients |
|---|---|---|
| 1 = Not a problem at all | 1 | 1% |
| 2 | 31 | 19% |
| 3 | 82 | 49% |
| 4 | 42 | 25% |
| 5 = Completely debilitating | 10 | 6% |

*Doctors did not answer the question for 3 of the patients.

As the results in Table 1 indicate, the average severity of the symptoms in the patients surveyed was 3.2 out of a scale of 1 to 5. The majority of the patients fell in the middle, where morning sickness was definitely a problem, but not completely debilitating. It is to be understood that doctors did not try any remedy with patients for whom morning sickness was “not a problem at all.”

The survey results indicate that most patients experienced some relief from the disclosed composition. For over half of the patients, whether they tried the composition alone or with other remedies, the composition gave them significant relief.

The doctors were asked how effective the composition was in treating the patient. Table 2 indicates the results of the answers to this question.

TABLE 2
Effectiveness of Composition*

| | Number of | Percent |
|---|---|---|
| Response | Patients | of Patients |
| Extremely effective - eliminated symptoms | 25 | 15% |
| Quite effective - symptoms significantly | 70 | 43% |
| reduced | | |
| Somewhat effective - symptoms lessened | 56 | 34% |
| Ineffective - no change | 13 | 8% |

*Doctors did not answer the question for five patients.

The doctors were next asked about the effectiveness of the composition for those patients who did not use another morning sickness remedy. Table 3 indicates the survey results for this question.

TABLE 3
Effectiveness of Composition Without Another Remedy

| | Number of | Percent of |
|---|---|---|
| Response | Patients | Patients |
| Extremely effective - eliminated symptoms | 13 | 23% |
| Quite effective - symptoms significantly | 22 | 39% |
| reduced | | |
| Somewhat effective - symptoms lessened | 18 | 32% |
| Ineffective - no change | 3 | 5% |

*A doctor did not answer the question for one of the patients.

The surveyed doctors were asked how likely they were to recommend the composition to patients in the future. Table 4 depicts their answers.

TABLE 4
Likelihood of Doctors Recommending Composition in the Future

| Response | Number of Doctors | Percent of Doctors |
|---|---|---|
| Extremely likely | 35 | 71% |
| Somewhat likely | 14 | 29% |
| Neither likely nor unlikely | 0 | 0% |
| Somewhat unlikely | 0 | 0% |
| Extremely unlikely | 0 | 0% |

*One doctor did not answer the question.

The doctors also had an opportunity to provide remarks. Twelve doctors made the following comments (note that “Magic” is the tradename of the composition disclosed herein):
1. Thanks—is a savior to my patients.
2. Prior to this study we had several patients who were very pleased with product.
3. Great reports from patients.
4. Each patient is different. Some meds work for some patients. We try to use Magic first. We need it more readily available to a wide range of patients
5. One patient liked it so much that when the bottle was almost empty, she filled it up with water and the little Magic left and would use that watered down version. She said it still worked!
6. Wanted something more natural [why didn't try anything else].
7. Less expensive than Zofran®.
8. Good product!
9. We would love more samples.
10. We offer this to all our patients who suffer from morning sickness. We have had no complaints from our Moms. This is a great product.
11. I was impressed with the effectiveness of the product. The patients liked the fact that ingredients were natural.
12. It is nice to have an option for patients to try before recommending an Rx med, which many OB's are not comfortable with. We've had mixed results with Magic, but that's true with any med. We will continue to offer this as a first-line tx (i.e., treatment) option!! (explanation of abbreviation added)

The survey also asked about other known morning sickness remedies. The doctors indicated that many patients tried Vitamin B6, Ginger, Zofran® (ondansetron hydrochloride, commercially available from GlaxoSmithKline in Research Triangle Park, N.C.), Phenergan® (promethazine hydrochloride, commercially available from American Home Products Corp., New York, N.Y.), and twelve other remedies in addition to the composition disclosed herein. Some of the other remedies included Reglan® (metochlopramide, commercially available from Robins Co., Richmond, Va.), Emetrol® (dextrose, levulose, phosphoric acid, commercially available from Pfizer, Inc., New York, N.Y.), Mylanta® (calcium carbonate, magnesium hydroxide, commercially available from Johnson & Johnson, Merck Consumer Pharmaceuticals Co., Fort Washington, Pa.), Premesis Rx® (calcium carbonate, vitamin B12, folic acid, vitamin B6, commercially available from KV Pharmaceutical Co., St. Louis, Mo.), Compazine® (prochlorperazine, commercially available from SmithKline Beecham, Philadelphia, Pa.), and meclizine (commercially available under the tradename Antivert® from Pfizer, Inc., New York, N.Y.). Table 5 illustrates which remedies, if any, the doctors tried besides the composition disclosed herein for each patient during the same pregnancy. Out of

TABLE 5
Remedies Tried in Addition to the Composition

| | Number of Patients | Percent of Patients |
|---|---|---|
| Remedy | That Tried Remedy | That Tried Remedy |
| Vitamin B6 | 55 | 36% |
| Ginger | 33 | 21% |
| Zofran | 33 | 21% |
| Phenergan | 25 | 16% |
| Compazine | 14 | 9% |
| Lemons, lemon tea | 6 | 4% |
| Premesis | 5 | 3% |
| Reglan | 5 | 3% |
| Prescription med (unspecified) | 3 | 2% |
| Meclizine | 2 | 1% |
| Antacids | 2 | 1% |
| Peppermint | 2 | 1% |
| Emetrol | 1 | 1% |
| Mylanta | 1 | 1% |
| Promethazine | 1 | 1% |
| Nothing else | 57 | 37% |

169 patients, 57 tried nothing else. Table 5 specifically depicts what remedies the other 112 patients tried, either alone or in combination with other products.

Table 5indicates that of those patients that tried at least one other remedy besides the composition disclosed herein, 87% of them found the composition to be more effective than at least one other remedy. Some of the patients tried more than one of the above remedies. Regardless of whether they tried one or more remedies besides the disclosed composition, each doctor was asked, for each patient: did the composition work 1) better than other products or combinations, 2) better than some, but not as well as others, or 3) not as well as other products or combinations. The overall results are shown in Table 6.

TABLE 6
Effectiveness of the Composition Compared to Other Remedies*

| | Number of | Percent of |
|---|---|---|
| Composition worked . . . | Patients | Patients |
| Better than other products or | 49 | 47% |
| combinations | | |
| Better than some, but not as well as | 42 | 40% |
| others | | |
| Not as well as other products or | 14 | 13% |
| combinations | | |

*57 patients did not try anything else, and doctors did not answer the question for 7 others.

In 13% of the cases for which there was an answer, the composition did not work as well as the other remedies that patients tried.

Overall, the results were mixed when the composition was compared to other remedies. Patients seemed to like the composition better than Vitamin B6 and ginger. However, patients seemed to prefer the more expensive prescription drugs Zofran ® and Phenergan®).

Another question in the survey asked the doctors what products each patient took besides the composition, and then whether the composition worked 1) better than other products or combinations, 2) better than some, but not as well as others, or 3) not as well as other products or combinations. Table 7 depicts the results of how the composition compared to each other remedy, whether the composition and the other remedy were both tried (without another remedy), or in combination with other remedies. As an example, some of the 55 patients who tried vitamin B6 and the composition may also have tried another remedy.

TABLE 7
Comparison of the Composition with other Morning Sickness Remedies
(The two taken alone or with other Remedies)

| | | | Percent of | |
|---|---|---|---|---|
| | | Percent of | Patients for | |
| | | Patients for | whom the | Percent of |
| | Number | whom the | Composition | Patients where |
| | of | Composition | Did | Doctor did not |
| | Patients | worked | Not Work | indicate a clear |
| Remedy | Taking | Better | as Well | comparison |
| Vitamin B6 | 55 | 45% | 11% | 44% |
| Ginger | 33 | 45% | 21% | 33% |
| Zofran ® | 33 | 36% | 42% | 21% |
| Phenergan ® | 25 | 40% | 32% | 28% |
| Compazine ® | 14 | 43% | 29% | 29% |
| Lemons, lemon | 6 | 100% | 0% | 0% |
| tea | | | | |
| Premesis ® | 5 | 40% | 0% | 60% |
| Reglan ® | 5 | 0% | 60% | 40% |
| Unspecified | 3 | 0% | 100% | 0% |
| Prescription | | | | |
| medicine | | | | |
| Meclizine ® | 2 | 100% | 0% | 0% |
| Tums ® | 2 | 100% | 0% | 0% |
| Peppermint | 2 | 50% | 0% | 50% |
| Emetrol ® | 1 | 0% | 0% | 100% |
| Mylanta ® | 1 | 100% | 0% | 0% |
| Promethazine ® | 1 | 0% | 100% | 0% |

*The one patient for whom the composition did not work as well as Vitamin B6 took the B6 via injection.

Table 8 depicts the results for those patients who took the composition in addition to the specific combination(s) of other remedies noted in the Table.

TABLE 8
Comparison of the Composition with other Morning Sickness Remedies

| Remedy/Remedies | | |
|---|---|---|
| Patient tried in | Number of | |
| addition to the | Patients Trying | |
| Composition | Combination | Results |
| Zofran ® only | 16 | The composition definitely worked |
| | | better for 5 patients |
| Ginger and B6 | 15 | The composition definitely worked |
| | | better than both for 7 patients |
| B6 only | 9 | The composition definitely worked |
| | | better for 7 patients |
| Phenergan ® only | 9 | The composition definitely worked |
| | | better for 4 patients |
| B6 and Zofran ® | 8 | The composition definitely worked |
| | | better for 1 patient |
| Ginger, B6 and | 8 | The composition definitely worked |
| Phenergan ® | | better for 2 patients |
| Compazine ® only | 6 | The composition definitely worked |
| | | better for 3 patients |
| Premesis ® only | 5 | The composition definitely worked |
| | | better for 2 patients |
| Ginger only | 3 | The composition definitely worked |
| | | better for 2 patients |

*This Table is indicative of the number of patients who took that combination, and their doctor said that the composition worked “Better than other products or combinations”.

It is believed that the small number of sample sizes that were given to doctors is not sufficient to draw any conclusions about specific combinations, but the results do support that the survey did not unearth any combination that conclusively works better than the composition disclosed herein all the time.

The overall results of the doctor survey indicate that 92% of patients found the composition disclosed herein at least somewhat effective, 15% found it extremely effective, and 43% found it quite effective. These results are in spite of the comments of six doctors who indicated that some patients remarked about an undesirable taste of the composition.

All of the doctors surveyed (except one who did not answer the question) indicated that they would recommend the composition to patients. Specifically, 71% were “extremely likely” to recommend it in the future, and 29% were somewhat likely to recommend it in the future.

The composition disclosed herein compared favorably to each of the other remedies, at least for some of the patients. Overall, 87% of the patients found the disclosed composition more effective than at least one other remedy. The composition disclosed herein compared most favorably to Vitamin B6 (the composition worked better at least 45% of the time), and least favorably to Zofran® (Zofran worked better at least 36% of the time).

While several embodiments have been described in detail, it will be apparent to those skilled in the art that the disclosed embodiments may be modified. Therefore, the foregoing description is to be considered exemplary rather than limiting.

What is claimed is:

1. An herbal composition for treating morning sickness, the herbal composition comprising vitamin B6, folic acid, ginger root extract, and red raspberry leaf.

2. The herbal composition as defined in claim 1, further comprising an additional ingredient selected from water, fructose, natural flavors, citric acid, beta carotene coloring, potassium sorbate, sodium benzoate, chamomile, peppermint, spearmint, papaya, catnip, squaw vine, rice flour, magnesium stearate, silica dioxide, and combinations thereof.

3. The herbal composition as defined in claim 1 wherein about 15 mg to about 35 mg of the vitamin B6 is present in about 15 mL of the composition.

4. The herbal composition as defined in claim 1 wherein about 100 mcg to about 800 mcg of the folic acid is present in about 15 mL of the composition.

5. The herbal composition as defined in claim 1 wherein about 10 mg to about 100 mg of the ginger root extract is present in about 15 ml of the composition.

6. The herbal composition as defined in claim 1 wherein about 5 mg to about 35 mg of the red raspberry leaf is present in about 15 ml of the composition.

7. The herbal composition as defined in claim 1 wherein the composition is in the form of at least one of liquid, powder, tablets, tea, capsules, or gelatin capsules.

8. The herbal composition as defined in claim 1 wherein the vitamin B6 is replaced with an ingredient selected from doxylamine succinate, dicyclomine, ondansetron, prochloroperazine, ginger root extract, peppermint, Chinese herbs, and combinations thereof.

9. The herbal composition as defined in claim 1 wherein the ginger root extract is replaced with an ingredient selected from doxylamine succinate, dicyclomine, ondansetron, prochloroperazine, vitamin B6, peppermint, catnip, licorice root, cayenne, Chinese herbs, and combinations thereof.

10. An herbal composition for treating morning sickness, the herbal composition comprising:
about 25 mg of vitamin B6;
about 200 mcg of folic acid;
about 50 mg of ginger root extract; and
about 20 mg of red raspberry leaf.

11. The herbal composition as defined in claim 10, further comprising an additional ingredient selected from water, fructose, natural flavors, citric acid, beta carotene coloring, potassium sorbate, sodium benzoate, chamomile, peppermint, spearmint, papaya, catnip, squaw vine, rice flour, magnesium stearate, silica dioxide, and combinations thereof.

12. The herbal composition as defined in claim 10 wherein the composition is in the form of at least one of liquid, powder, tablets, tea, capsules, or gelatin capsules.

13. A method for treating morning sickness, the method comprising administering to a patient an herbal composition, the composition including a mixture of effective amounts of each of vitamin B6, folic acid, ginger root extract, and red raspberry leaf.

14. The method as defined in claim 13 wherein the composition further includes an additional ingredient selected from water, fructose, natural flavors, citric acid, beta carotene coloring, potassium sorbate, sodium benzoate, chamomile, peppermint, spearmint, papaya, catnip, squaw vine, rice flour, magnesium stearate, silica dioxide, and combinations thereof.

15. The method as defined in claim 13 wherein about 15 mg to about 35 mg of the vitamin B6 is present in about 15 mL of the composition.

16. The method as defined in claim 13 wherein from about 100 mcg to about 800 mcg of the folic acid is present in about 15 mL of the composition.

17. The method as defined in claim 13 wherein from about 10 mg to about 100 mg of the ginger root extract is present in about 15 ml of the composition.

18. The method as defined in claim 13 wherein from about 5 mg to about 35 mg of the red raspberry leaf is present in about 15 ml of the composition.

19. The method as defined in claim 13 wherein the composition is in the form of at least one of liquid, powder, tablets, tea, capsules, or gelatin capsules.
